(19) United States
(12) Patent Application Publication
(10) Pub. No.: US 2026/0080686 A1
(43) Pub. Date: Mar. 19, 2026

(54) DETECTION AND COUNTING APPARATUS FOR ECOLOGICAL ENVIRONMENTS

(71) Applicant: University of Florida Research Foundation, Inc., Gainesville, FL (US)

(72) Inventors: Nathan S. Boyd, Gainesville, FL (US); Renato Furlanetto, Gainesville, FL (US); Arnold W. Schumann, Gainesville, FL (US)

(21) Appl. No.: 19/332,520

(22) Filed: Sep. 18, 2025

Related U.S. Application Data
(60) Provisional application No. 63/696,553, filed on Sep. 19, 2024.

(51) Int. Cl. G06V 20/52; A01K 67/366; A01M 21/04; G01N 33/00; G06V 10/70; G06V 20/10; G06V 40/10; H04N 7/18; H04N 23/695

(52) U.S. Cl. CPC G06V 20/52; G01N 33/0098; G06V 10/70; G06V 20/188; G06V 40/10; H04N 7/188; H04N 23/695; A01K 67/366; A01M 21/043

(57) ABSTRACT

Disclosed are various embodiments for a data collection apparatus that is configured to detect, count, and/or identify organisms and their characteristics from a media item (e.g., an image, a video, etc.) of one or more ecological environments. For example, a system can include a camera for capturing an image of an ecological environment and a computing device. The computing device can be configured to at least identify a triggering condition for the ecological environment and capture, using the camera, an image of the ecological environment based at least in part on the triggering condition. The computing device can determine a characteristic of an organism in the ecological environment based at least part in a machine learning model.

### CROSS-REFERENCE TO RELATED APPLICATIONS

This application claims the benefit of and priority to U.S. Provisional Application No. 63/696,553, filed Sep. 19, 2024, entitled “Detection and Counting Apparatus for Ecological Environments,” the entire contents of which is hereby incorporated herein by reference.

### STATEMENT REGARDING FEDERALLY SPONSORED RESEARCH OR DEVELOPMENT

This invention was made with government support under Grant No. 20236701339898, awarded by the United States Department of Agriculture, National Institutes of Food & Agriculture. The government has certain rights in the invention.

### BACKGROUND

Often, ecological models can be based on data collected manually by individuals. Trained individuals have to be knowledgeable in identifying particular species and their characteristics. In some cases, these individuals have to regularly visit the same ecological environments to in order to track the development of the species over a period of time. As such, the data collection process for formulating ecological models can be time-consuming and labor-intensives for trained individuals.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1A is a drawing depicting a side view of a data collection apparatus according to various embodiments of the present disclosure.

FIG. 1B is a drawing depicting a front view of the data collection apparatus of FIG. 1 according to various embodiments of the present disclosure.

FIG. 2 is a drawing of a network environment according to various embodiments of the present disclosure.

FIG. 3 is a flowchart illustrating one example of functionality implemented as portions of an application executed in a computing environment in the network environment of FIG. 2 according to various embodiments of the present disclosure.

### DETAILED DESCRIPTION

Disclosed are various approaches for a data collection apparatus that is configured to detect, count, and/or identify organisms and their characteristics from a media item (e.g., an image, a video, etc.) of one or more ecological environments, such as agricultural fields, forests, grasslands, and other suitable ecological environments.

Typically, ecological models can be based on data collected manually by trained individuals. These individuals are trained to identify particular species and their characteristics. In some cases, these individuals regularly visit this the same ecological environment in order to track the development of species over a period of time. As such, the data collection process for formulating ecological models can be time-consuming and labor-intensive for trained individuals. The collected data can be used to generate ecological models, and the ecological models can be used for various purposes. For example, within the field of agriculture, ecological models can provide weed emergence data, and the weed emergence data can be used for cultivation, herbicide application, determining an appropriate time for seed planting, and other suitable agriculture purposes. As another example, within the field of insect farming, ecological data can provide insights for the development of honeybees, silkworms, crickets, waxworms, and other suitable insects. This insect development data can be used for raising and/or breeding insects for insect products (e.g., honey, beeswax, silk, insects for animal feed, etc.).

Accordingly various embodiments of the present disclosure relate to an improvement in the field of computer vision by using a computing device equipped with a camera to identify organism characteristics from an image or a video. With regard to weed plant development, for example, the various embodiments can identify, from an image or a video frame, a particular weed species, count the number of weed plants in the image or video frame, and identify other plant development characteristics. Additionally, the various embodiments can collect soil moisture data and air temperature data for the ecological environment associated with the weed plant characteristics. The ecological environment data can be associated with identified organism characteristics. Additionally, the collected data can be transmitted to one or more remote computing devices.

Further, the various embodiments of the present disclosure include a data collection apparatus that can be uniquely configured to collect data in an ecological environment over long periods of time. The data collection apparatus includes a computing device executing an artificial intelligence machine learning model that has been trained to identify one or more organism characteristics in one or more ecological environments. For example, training data can be used by a machine learning algorithm to generate the artificial intelligence machine learning model. The training data can include data related to various organism characteristics.

The various embodiments of the present disclosure provide multiple advantages over existing methods of collecting organism data. For example, the various embodiments enable autonomous, remote monitoring of organisms in an ecological environment. Capturing data in ecological environments over long periods of time can present challenges dealing with the weather (e.g., sun, heat, humidity, wind gust, etc.) and agricultural vehicle traffic. As such, various environment elements can affect the ability of the data collection apparatus to collect data over long periods of time in a remote location. The data collection apparatus can be configured for various settings related to capturing data, such as data collection frequency, camera settings, sensor settings, and other suitable settings.

Further, the various embodiments can include a camera attached to a structural member which can be moved by a motor, an actuator, and other suitable mechanical devices. These mechanical devices can be used to autonomously position the camera for an image capture of an organism in the ecological environment. In some examples, the camera can be mechanically moved after an image capture in order to avoid collisions with other objects (e.g., agricultural vehicle traffic, animals, etc.) in the environment. Further, the various embodiments of the present disclosure have been configured to withstand weather conditions for various outdoor environments.

In some examples, the various embodiments of the data collection apparatus can be remotely controlled and operated by a remote computing device (e.g., a client device, a computing environment, a server). The remote computing device can transmit instructions and/or commands for the data collection apparatus. For example, the instructions can include triggering conditions, timing intervals for image capturing intervals, instructions for moving or positioning merchant devices/components of the data collection apparatus.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principals disclosed by the following illustrative examples.

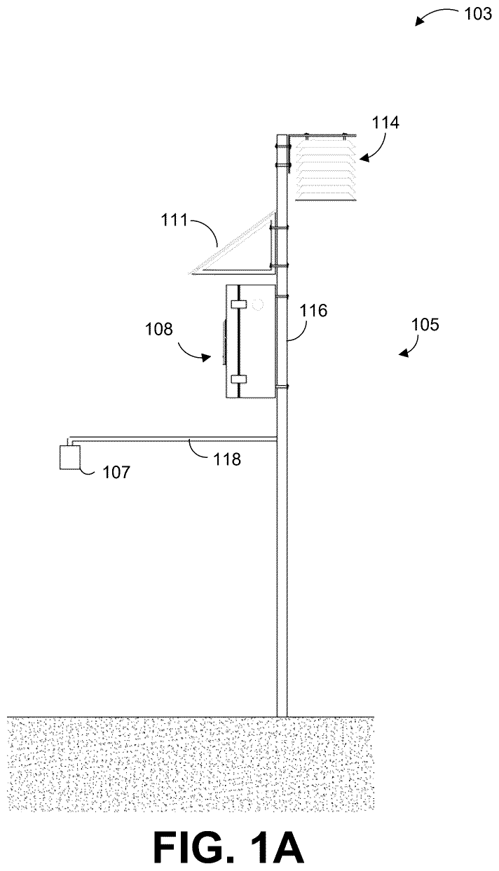

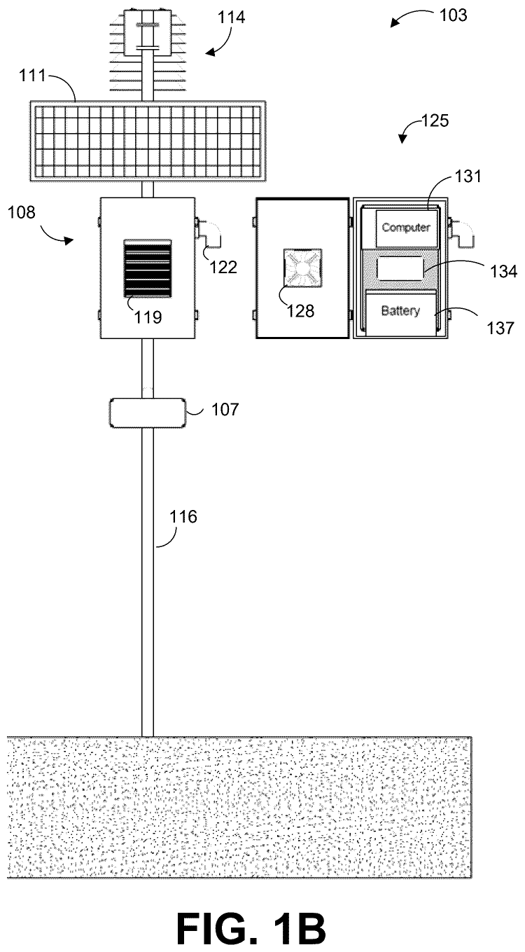

As illustrated in FIG. 1A, shown is a side view of a data collection system 103 for autonomously and remotely capturing image data and determining organism characteristics over a period of time. The data collection system 103 comprises a fixed structure 105, a camera 107, a device enclosure 108, a solar panel 111, a sensor 114, and other suitable components. In depicted FIG. 1, the fixed structure 105 comprises a rod 116, an arm 118, and other suitable components.

The fixture structure 105 can be used as an apparatus for supporting the various components of the data collection system 103. The device enclosure 108, the arm 118, the solar panels 111, the sensors 114, and other suitable components can be attached to the rod 116. The components can be attached to the fixture structure 105 according to other arrangements.

The camera 107 can be used for capturing images and video for object detection and processing. In some examples, the camera 107 can include specifications such as high resolution (e.g., 4 k resolution/4,000 pixels or higher), Ingress Protection of at least 65 (IP65), or other suitable cameras settings for an outdoor environment.

In some examples, the camera 107 can be a three-dimensional camera 107 that can be used to also capture depth data relating to the detected organism objects. The depth data can be used to determine the height of a plant. In some other examples, instead of a three-dimensional camera 107, a second high resolution camera 107 is used. The second high resolution camera 107 can be situated at a different position from a first high resolution camera 107. The second high resolution camera 107 can be positioned in order to determine a depth of the targeted organism object.

The device enclosure 108 can be a container for housing electrical and computing components used to operate the data collection system 103. The device enclosure 108 protects the electrical and computing components from the weather and other elements in the ecological environments. The device enclosure 108 can be opened and closed, in which the opened state allows for user access to the interior of the device enclosure 108.

The solar panels 111 can be used to collect solar energy from the sun and provide power to the device enclosure 108. In some examples the solar panels 111 can 111 can be equipped with a motor for mechanically moving panels in order to optimize solar energy harvesting. In some examples, the motor can be in data communication with a computing device within the device enclosure 108.

The sensors 114 can be used to collect weather data (e.g., temperature, wind, humidity, etc.). In some examples, the rod 116 can have a soil sensor attached for collecting soil measurements for the ecological environment. In some implementations, the soil sensor can be a separate component that is in data communication with a computing device within the device enclosure 108. Other sensors can be used to collect other ecological environment data.

The rod 116 can be an elongated structural member that can be inserted into the ground. In some examples, one end of the rod can be tapered in order to facilitate an insertion into the ground. The shape of the rod 116 can vary.

The arm 118 can be attached to the camera 107. In some implementations, the arm 118 is a structural member. In some examples, the arm 118 can be attached to a mechanical device, in which the mechanical device can mechanically moving the arm 118. During an image capture, the arm 118 can be in an image capture orientation or a first position. In other examples, the arm 118 can be moved to an unobstructed orientation or a second point in order to avoid collisions with other objects, such as vehicles, people, animals, and other suitable. Additionally, the arm 118 can be moved in order to minimize the effect of weather conditions. For instance, in an extended position the wind and rain may have an increased force for knocking over or moving the data collection system 103.

The mechanical device can be a motor (e.g., a stepper motor), an actuator and other suitable mechanical devices. The mechanical device can be electrically activated in order to move the arm 118 in one or more dimensions (e.g., one, two, or three dimensions). The mechanical device can be electrically activated in order to rotate or translate the arm 118. For example, the mechanical device can be activated in order to raise the arm 118 from a lower position to a higher position for capturing a media item. In another example, the mechanical device can be activated to extend the arm 118 horizontally in order to expand the length of the arm 118 for an image capture of the ecological environment. In some examples, the mechanical device can move the arm 118 in six axes of motion or three-dimensional space.

With reference to FIG. 1B, shown is a front view of the data collection system 103. As shown in the depicted in FIG. 1B, the device enclosure 108 has a vent 119 on the exterior side of the device enclosure 108. The side of the device enclosure 108 has an access port 122.

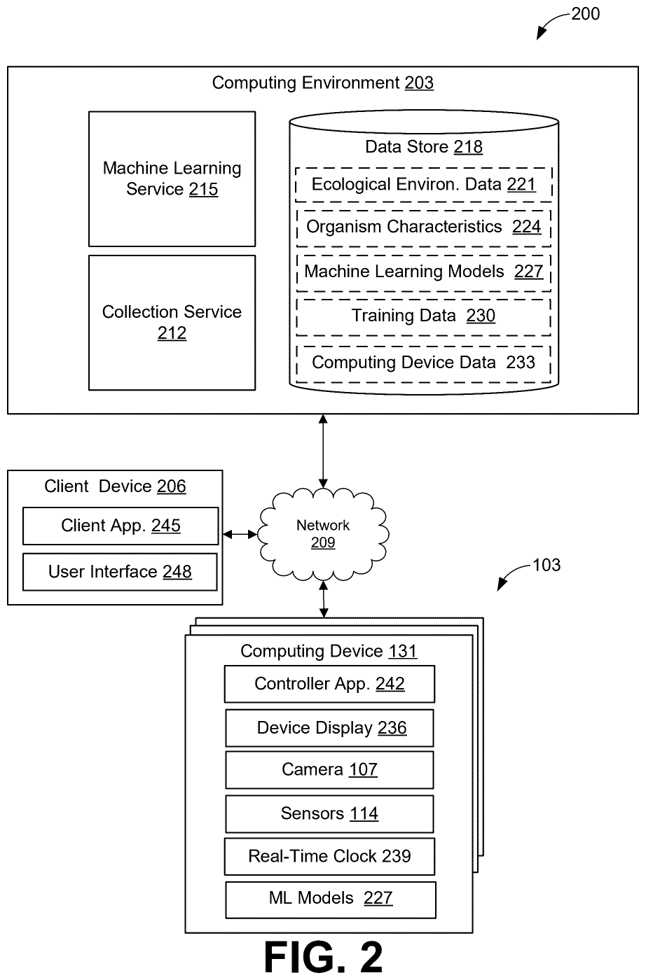

Reference 125 refers to the device enclosure 108 in an opened state. Within the device enclosure 108, a fan 128 is situated on an enclosure door. The device enclosure 108 includes a computing device 131 (labeled as “Computer” in FIG. 1B), a solar charger 134, and a battery 137. The computing device 131 can represent a computing processing unit or a controller used to operate the components of the data collection system 103. In some examples, a single board computer, such as Raspberry PI®, Nvidia's Jetson Nano®, a controller, or other suitable single board computers. The computing device 131 will be described in further detail in FIG. 2.

The computing device 131 can be electrically coupled to the solar charger 134, the battery 137, the fan 128, the camera 107, the sensors 114, the solar panel 111, and other suitable components. The computing device 131 can transmit (e.g. control signals, settings, instructions, etc.) and receive data to these components.

The solar charger 134 can be used to recharge the battery 137 with power provided by the solar panel 111. In some examples, the solar charger 134 can perform a power conversation in order to provide power at desired power levels (e.g., current, voltage) for the battery and other components. In some examples, the solar charger 134 can be instructed (via. The controller device 131) to provide power directly to one or more components of the data collection system 103.

The battery 137 can store power (e.g., voltage) for the data collection system 103. The battery 137 can be electrically coupled to the solar charger 134, the computing device 131, the fan 128, the camera 107, the sensors 114, the solar panel 111, and other suitable components. In some examples, the battery 137 can provide direct current (DC) to these components. In some instances, these components may not be electrically coupled to the battery and may be powered by other methods.

With reference to FIG. 2, shown is a network environment 200 according to various embodiments. The network environment 200 can include a computing environment 203, a computing device 131 (e.g., for operating the data collection system 103), and a client device 206, which can be in data communication with each other via a network 209.

The network 209 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 209 can also include a combination of two or more networks 209. Examples of networks 209 can include the Internet, intranets, extranets, virtual private networks (VPNs), and similar networks.

The computing environment 203 can include one or more computing devices that include a processor, a memory, and/or a network interface. For example, the computing devices can be configured to perform computations on behalf of other computing devices or applications. As another example, such computing devices can host and/or provide content to other computing devices in response to requests for content.

Moreover, the computing environment 203 can employ a plurality of computing devices that can be arranged in one or more server banks or computer banks or other arrangements. Such computing devices can be located in a single installation or can be distributed among many different geographical locations. For example, the computing environment 203 can include a plurality of computing devices that together can include a hosted computing resource, a grid computing resource or any other distributed computing arrangement. In some cases, the computing environment 203 can correspond to an elastic computing resource where the allotted capacity of processing, network, storage, or other computing-related resources can vary over time.

Various applications or other functionality can be executed in the computing environment 203. The components executed on the computing environment 203 include a collection service 212, a machine learning service 215, and other applications, services, processes, systems, engines, or functionality not discussed in detail herein.

The collection service 212 can be executed to collect and provide instructions to one or more computing devices 131 of the data collection system 103. In some examples, the collection service 212 can generate reports and ecological models (e.g., plant models, insect models, etc.) based at least in part on the collected data received from one or more data collections systems 103. The machine learning service 215 can be executed to train, evaluate, validate, deploy machine learning models to data collection systems 103, and other suitable machine learning functions.

Also, various data is stored in a data store 218 that is accessible to the computing environment 203. The data store 218 can be representative of a plurality of data stores 218, which can include relational databases or non-relational databases such as object-oriented databases, hierarchical databases, hash tables or similar key-value data stores, as well as other data storage applications or data structures. Moreover, combinations of these databases, data storage applications, and/or data structures may be used together to provide a single, logical, data store. The data stored in the data store 218 is associated with the operation of the various applications or functional entities described below. This data can include ecological environment data 221, organism characteristics 224, machine learning models 227, training data 230, computing device data 233, and potentially other data.

The ecological environment data 221 can represent data collected about the ecological environment around the location of a particular data collection system 103. The ecological environment data 221 can include sensor measurement data (e.g., soil moisture, air temperature, humidity, wind gust, precipitation, etc.).

The organism characteristics 224 can represent characteristics determined or identified by the data collection systems 103 from the images and/or video captured in the ecological environment. Some non-limiting examples of organism characteristics 224 for plants can include species identified in the media items (e.g., image, video), a count of the species in the media, a leaf count (e.g., a number of leaves on a plant), leaf characteristics, plant height, growth characteristics (e.g., stage of development), heath indicators (e.g., healthy indicators, disease indicators), and other suitable plant characteristics 224.

Some non-limiting examples of organism characteristics 224 for insects can include species identified in the media item, a count of the species in the media, growth characteristics (e.g., stage of development), heath indicators (e.g., healthy indicators, disease indicators), movement or activity indicators, location indicators, and other suitable insect characteristics 224.

The machine learning models 227 can represent data associated with machine learning models that have been trained for deployment. Machine learning algorithms can be employed to generate machine learning models 227 based at least in part on training data 230 (e.g., training datasets, validation data, preprocessing data, raw data, etc.). In some examples the machine learning algorithms can include object detection algorithms, such as histogram of oriented gradients, Region-based Convolutional Neural Networks (R-CNN), Faster R-CNN, Single Shot Detector, and other suitable object detection algorithms.

In some examples, a first set of machine learning models 227 can be used for object detection of organisms in the media item. The first set of machine learning models 227 can include one or more machine learning models 227 that have been generated with a machine learning algorithm. These trained first set of machine learning models 227 can have weights and parameters that have been determined from the training data 230 for detecting organism objects in a media item. These first set of machine learning models 227 can be provided one or more media items as input, and these first set of machine learning models 227 can provide output in the form of one or more identified organism objects.

In some examples, a second set of machine learning models 227 can be used for identifying organism characteristics 224. The second set of machine learning models 227 can include one or more machine learning models 227 that have been generated with a machine learning algorithm. These trained second set of machine learning models 227 can have weights and parameters that have been determined from the training data 230 for identifying organism characteristics 224 of an identified organism object in a media item. These second set of machine learning models 227 can be provided one or more identified organism objects as input, and these second of machine learning models 227 can provide output in the form of one or more organism characteristics for the identified organism.

The training data 230 can represent data used for generating the machine learning models. The training data 230 can include data sets for each of the organism characteristics 224 (e.g., species identification, a species count, a leaf count, growth characteristics, plant height, heath indicators, etc.). The datasets can include a pair of valid indicator examples and invalid indicator examples. For example, the dataset can include valid detection of a weed species and invalid detection of the weed species. In another example, the dataset can include valid detection of five weed species counted and invalid detection of five weed species counted. The training data 230 can include other data related to training, evaluating, preprocessing, raw data, feature extraction data, and other suitable training data 230.

The computing device data 233 can include data associated with each data collection systems 103 located in one or more ecological environments. The computing device data 233 can be a location, a proximity to other computing devices 131, networking data (e.g., Internet Protocol address, cellular communication data), and other suitable data.

The computing device 131 is representative of a computing processing device operating a data collection system 103. The computing device 131 can include a processor-based system such as a computer system. Such a computer system can be embodied in the form of a single board computer multiple board computer, a mobile computing device, an application-specific integrated circuit for an artificial neural network (e.g., objection detection machine learning neutral networks), or other devices with like capability. The computing device 131 can include one or more processing units, which may include hardware accelerators (e.g., Graphics Processing units, Vision Processing units, Field-Programmable Gate Array, Application-Specific Integrated Circuits) for object detection or computer vision algorithms. The computing device 131 can include one or more device displays 236, such as liquid crystal displays (LCDs), electrophoretic ink (“E-ink”) displays, or other types of display devices. In some instances, the display can be a component of the computing device 131 or can be connected to the computing device 131 through a wired or wireless connection.

The computing device 131 can include various components. For example, the computing device 131 can include the camera 107, the sensor 114, a real-time clock 239, a processor, memory, a transceiver device, and other suitable components. The real-time clock 239 can be an electrical component for tracking time. In some examples, the real-time clock 239 can generate a clock signal (e.g., for higher accuracy and better consistency) for the computing device 131. The clock signal can be used to determine when the camera 107 should capture media (e.g., image or video) of the ecological environment. The transceiver device can be used for wireless data communication over network 209 or local networks. Some non-limiting examples of wireless data communication protocols that be executed by the transceiver device include Wi-Fi protocol, a BLUETOOTH® protocol, a cellular protocol, and other suitable wireless data communication protocols. The computing device 131 can also include other components such as memory, a camera port, and suitable components.

The computing device 131 can be configured to execute various applications such as a controller application 242 or other applications. The controller application 242 can be executed to control the execution of various tasks by the components of the data collection system 103. For example, the controller application 242 can instruct the camera 107 to capture images or video of the ecological environment on a timed scheduled (e.g., every day at 8:00 AM, noon, and 4:00 PM, etc.), upon a request, or based at least in part on other triggering conditions. The controller application 242 can analyze the capture media (e.g., images, video) to identify organism characteristics 242 (e.g., plant characteristics, insect characteristics, etc.) based at least in part on one or more machine learning models 227. The controller application 242 can manage the collection of ecological environment data 221 (e.g., sensor measurements). The controller application 242 can transmit the ecological environment data 221 and the organism characteristics 242 to the collection service 212. In some examples, the controller application 242 can receive operating instructions (e.g., image capture positions, image capture timing intervals, unobstructed orientation positions, etc.) from the client application 245 and/or the collection service 212. Thus, the data collection system 103 (e.g., via the controller application 242) can be remotely controller by a remoting computing device (e.g., the client device 206, the computing environment 203).

The client device 206 is representative of a plurality of client devices that can be coupled to the network 209. The client device 206 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, and similar devices), or other devices with like capability. The client device 206 can include one or more displays, such as liquid crystal displays (LCDs), gas plasma-based flat panel displays, organic light emitting diode (OLED) displays, electrophoretic ink (“E-ink”) displays, or other types of display devices. In some instances, the display can be a component of the client device 206 or can be connected to the client device 206 through a wired or wireless connection.

The client device 206 can be configured to execute various applications such as a client application 245 or other applications. The client application 245 can be executed in a client device 206 to access network content served up by the computing environment 203 or other servers, thereby rendering a user interface 248 on the display. To this end, the client application 245 can include a browser, a dedicated application, or other executable, and the user interface 248 can include a network page, an application screen, or other user mechanism for obtaining user input. The client device 206 can be configured to execute applications beyond the client application 245 such as email applications, social networking applications, word processors, spreadsheets, or other applications. The client application 245 can be executed to provide instructions or settings to the computing device 131. Additionally, the client application 245 retrieve ecological environment data 221, organism characteristics 224, and other suitable data from the computing device 131

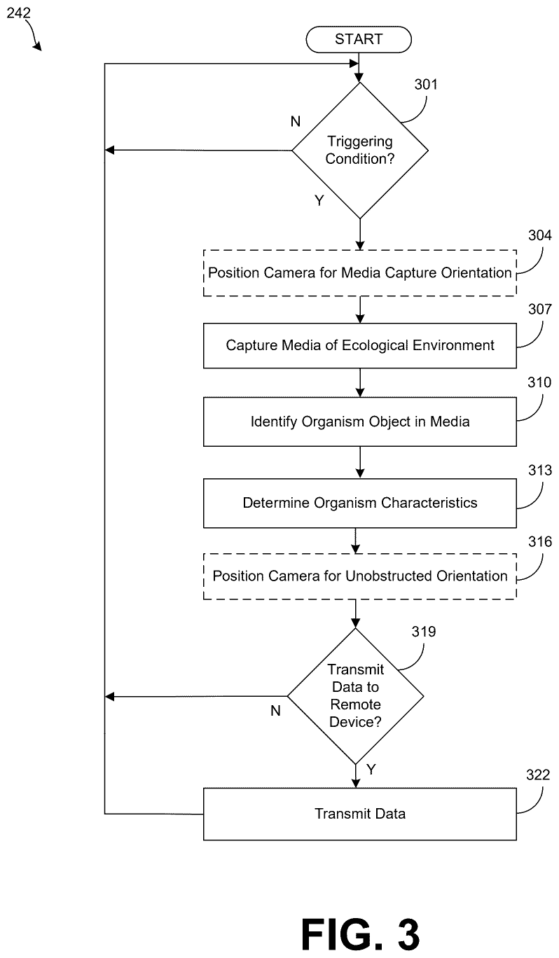

Referring next to FIG. 3, shown is a flowchart that provides one example of the operation of a portion of the controller application 242. The flowchart of FIG. 3 provides merely an example of the many different types of functional arrangements that can be employed to implement the operation of the depicted portion of the controller application 242. As an alternative, the flowchart of FIG. 3 can be viewed as depicting an example of elements of a method implemented within the network environment 200.

To begin, the machine learning service 215 can be used to generate machine learning models 227. Training data 230 can be prepared for the specific identification of organism characteristics 224. For example, a training dataset can be prepared for weed emergence, weed characteristics, and other suitable plant characteristics. For example, the training dataset for weed species can include pairs of each organism characteristic 224, such as a valid weed species and an invalid weed species; a valid count of weed leaves and an invalid count of weed leaves; a valid disease classification and an invalid disease classification; a valid weed height and an invalid weed height; and other suitable characteristics. After being generated, the client application 206 and/or machine learning service 215 can transmit the machine learning model 227 to the controller application 242 for the data collection system 103.

In block 301, the controller application 242 can identify a triggering condition for an ecological environment. In some instances, the computing device 131 can be in a low-power state (e.g. a sleeping state, a dormant state) in order to minimize the power consumption on the battery 137 while one or more triggering conditions are being evaluated. Some non-limiting examples of triggering conditions can include a timer, a time interval, an on-demand instruction from the client device 206 and/or the computing environment 203, a weather condition, a motion-detected event, and other suitable triggering conditions. If a triggering condition is identified, then the controller application 242 can activate to computing device 131 to an activate state and proceed to block 304. If a triggering condition is not identified, then the controller application 242 can proceed to block 301.

In one non-limiting example, with respect to a data collection system 103 for monitoring weed plants, the triggering condition can be a schedule that includes capturing a media item (e.g., an image, video, etc.) every eight hours. In another non-limiting example, with respect to a data collection system 103 for monitoring insect species, the trigger conditions can be a motion-detected event and/or a schedule for capturing a media item. In another non-limiting example, the triggering condition can be caused by a sensor measurement from a sensor 114. For instance, a temperature sensor 114 can provide temperature measurements that exceed a temperature threshold. A rain gauge sensor 114 can provide rain measurements that exceed a rain threshold. These weather measurements can be configured as a triggering condition.

In another non-limiting example, the data collection system 103 can receive instructions from a remote computing device, such as a client device 206, a computing environment 203, or other suitable devices. The remote computing device can transmit to the data collection system 103 a set of instructions or an operating sequence. Upon receiving the instructions, the data collection system 103 can execute the set of instructions. The instructions can include triggering conditions, movement instructions for media capture orientations/positions or unobstructed orientations/positions, timing interval for a frequency for capturing images or capturing video segments, and/or other suitable instructions. For example, the movement instructions for a stepper motor can include a direction signal for the movement of a structural member (e.g., the arm 119, the camera 107), a quantity of step pulses, a quantity of counting steps, a holding torque for providing a holding torque for resisting external forces in order to lock a motor in place. In another example, the movement instructions for an actuator can include a desired position signal (e.g., via a voltage signal, a pulse-width modulation signal, etc.) for representing the target position, a position feedback (e.g., via a position sensor, a potentiometer, a hall-effect sensor etc. for sending back signals indicating a current position), an error calculation, an adjusting movement, and/or other suitable instructions.

In block 304, the controller application 242 can position the camera 107 for a media capture orientation or a media capture position. The controller application 242 can command a mechanical device (e.g., a stepper motor, an actuator, etc.) to activate in order to move an arm 118 to a media capture orientation or position. In some examples, the arm 118 can be raised from a lowered position to a raised positioned. In other cases, the arm 118 can be extended laterally from a position near the rod 116 to an extended position further away from rod 116. Via the arm 118, the camera 107 can be manipulated in six different axes of orientation. Block 304 has a dashed line because it may be omitted in some embodiments.

In block 307, the controller application 242 can capture media (e.g., an image or a video) of the ecological environment using the camera 107. The controller application 242 can provide the camera 107 with an instruction or a command to capture media according to certain conditions, such as a timing interval and other conditions. The controller application 242 can provide camera settings such as resolution, shutter speed, frame rate, aperture, and other suitable camera settings.

In block 310, the controller application 242 can identify one or more organism objects in the media based at least in part on a first machine learning model (e.g., machine learning-based techniques, deep learning-based techniques, etc.). Some non-limiting examples of deep-learning based techniques can include regions with convolution neural network (R-CNN), Faster R-CNN, Mask R-CNN, and other suitable object detection techniques for generating machine learning models. These deep learning based techniques can include an encoder and a decoder. In some examples, the controller application 242 can generate a data collection file that stores the organism objects, the organism characteristics 224, and other suitable data.

In some examples, the controller application 242 can filter out detected objects that are identified as undesired organism objects, and other undesired objects. For example, the controller application 242 can be configured to identify weed plants in an ecological environment. The controller application 242 can filter out other agricultural plants that are identified in the media item. If all of the identified organism objects are filtered out, then the controller application 242 can put the computing device 131 in a low power state and proceed to block 301.

In some examples, the controller application 242 can identify a desired organism and can move/adjust the camera position based at least in part on the movement of the organism. For example, if a desired insect is identified, the controller application 242 can move or adjust the camera 107 in order to keep the insect with a viewing area for the camera 103.

In block 313, the controller application 242 can determine organism characteristics for the detected organism objects in the media based at least in part a second machine learning model. In some examples, the controller application 242 can identify organism characteristics 224 associated with the identified organism objects. In some examples, the identified organism objects are filtered for a certain organism object (e.g., a particular weed species) before an analysis of the organism characteristic 224 are generated. In some examples, the controller application 242 can generate a data collection file that stores the organism objects, the organism characteristics 224, and other suitable data.

In block 316, the controller application 242 can position the camera 107 for an unobstructed orientation or position. After the media (e.g., image or video) has been captured, the controller application 242 can instruct the arm 118 to move to an unobstructed position or orientation. In some examples, the controller application 242 can provide an instruction to the mechanical device to move the arm 118 to the unobstructed position or orientation.

In block 319, the controller application 242 can determine whether to transmit data (e.g., organism characteristics 224, ecological environment data 221) to the computing environment 203 based at least in part on one or more conditions. The data can be transmitted in a file, a data structure, and other suitable means for transmitting data. The controller application 242 can have conditions for transmitting the ecological environment data 221 and the organism characteristics 224. Some non-limiting examples of conditions can include a time schedule (e.g., every day at 8 PM), on demand, an event-based condition, and other suitable conditions. If data is to be transmitted, then the controller application 242 can proceed to block 322. If data is not to be transmitted, then the controller application 242 can proceed to block 301. In some examples, the controller application 242 can put the computing device 131 in a low power mode and then proceed to block 301.

In block 322, the controller application 242 can transmit the data to the remote computing device (e.g., via the client application 245 and/or the collection service 212). The controller application 242 can transmit the data based at least in part on networking settings for the client application 245 and/or the collection service 212. In some embodiments, a first computing device 131 of a first data collection system 103 can collect data from other computing devices 131 of other data collection systems 103. After aggregating data from multiple computing devices 1031, the first computing device 131 can transmit the aggregated data to the client application 245 and/or the collection service 212. In some examples, the controller application 242 can put the computing device 131 in a low power mode in order to minimize the power consumption on the battery 137. Then, the controller application 242 can proceed to block 301.

In some examples, after the remote computing device has receive the data from the data collection system 103, the remote computing device can provide updated instructions to the data collection system 103. For example, the remote computing device can analyze the data to determine whether the data meets a threshold (e.g., an integrity threshold, a volume threshold). If the threshold is met, the remote computing device can transmit updated instructions. The update instructions can include instructions to target a different location within the ecological environment, a different organism, or other suitable targets for analysis. In other examples, the updated instructions can include calibration instructions for the camera settings, movement positions setting or other suitable instructions for data collection.

A number of software components previously discussed are stored in the memory of the respective computing devices and are executable by the processor of the respective computing devices. In this respect, the term “executable” means a program file that is in a form that can ultimately be run by the processor. Examples of executable programs can be a compiled program that can be translated into machine code in a format that can be loaded into a random-access portion of the memory and run by the processor, source code that can be expressed in proper format such as object code that is capable of being loaded into a random-access portion of the memory and executed by the processor, or source code that can be interpreted by another executable program to generate instructions in a random-access portion of the memory to be executed by the processor. An executable program can be stored in any portion or component of the memory, including random-access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory includes both volatile and nonvolatile memory and data storage components. Volatile components are those that do not retain data values upon loss of power. Nonvolatile components are those that retain data upon a loss of power. Thus, the memory can include random-access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM can include static random-access memory (SRAM), dynamic random-access memory (DRAM), or magnetic random-access memory (MRAM) and other such devices. The ROM can include a programmable read-only memory (PROM), an erasable programmable read-only memory (EPROM), an electrically erasable programmable read-only memory (EEPROM), or other like memory device.

Although the applications and systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 4 shows the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowchart of FIG. 4 shows a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowchart of FIG. 4 can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that includes software or code can be embodied in any non-transitory computer-readable medium for use by or in connection with an instruction execution system such as a processor in a computer system or other system. In this sense, the logic can include statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. Moreover, a collection of distributed computer-readable media located across a plurality of computing devices (e.g, storage area networks or distributed or clustered filesystems or databases) may also be collectively considered as a single non-transitory computer-readable medium.

The computer-readable medium can include any one of many physical media such as magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random-access memory (RAM) including static random-access memory (SRAM) and dynamic random-access memory (DRAM), or magnetic random-access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device, or in multiple computing devices in the same computing environment 300.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A system, comprising:
a fixed structure;
a camera that is attached to the fixed structure, the camera targeting an ecological environment;
a computing device that is attached to the rod, the computing device comprising a processor, and a memory, the computing device being in data communication with the camera; and
machine-readable instructions stored in the memory that, when executed by the processor, cause the computing device to at least:
identify a triggering condition for the ecological environment;
capture, using the camera, an image of the ecological environment based at least in part on the triggering condition; and
determine a characteristic of an organism in the ecological environment based at least part in a machine learning model, the machine learning model using an object detection technique and the machine learning model being trained with a dataset for identifying the characteristic of the organism.

2. The system of claim 1, wherein the organism is a plant or an insect.

3. The system of claim 1, wherein the machine-readable instructions further cause the computing device to at least:
actuate a mechanical device for moving a camera arm of the camera into an image capture orientation based at least in part on the triggering condition being detected, the camera arm being attached to the fixed structure.

4. The system of claim 1, wherein the organism is a weed plant and the characteristic comprises at least one of: a weed species type, a quantity of weeds in the image; a quantity of weed leaves on a respective weed plant, or a growth stage of the respective weed plant.

5. The system of claim 1, wherein the triggering condition is a scheduled image capture time based at least in part on an interval time.

6. The system of claim 1, wherein the machine-readable instructions further cause the computing device to at least:
actuate a mechanical device for moving a camera arm of the camera to an unobstructed orientation based at least in part on an occurrence of the capture of the image of the ecological environment having been completed, the camera arm being attached to the fixed structure.

7. The system of claim 6, wherein mechanical device is at least one of a stepper motor or an actuator.

8. The system of claim 1, wherein the machine-readable instructions further cause the computing device to at least:
transmit the characteristic of the organism to a remote computer device based at least in part on a transmission condition.

9. A method of operating a data collection system for identifying organism characteristics, comprising:
identifying, by a computing device, a triggering condition for the ecological environment;
capture, using a camera in communication with the computing device, an image of the ecological environment based at least in part on the triggering condition; and
determining, by the computing device, a characteristic of an organism in the ecological environment based at least part in a machine learning model, the machine learning model using an object detection technique and the machine learning model being trained with a dataset for identifying the characteristic of the organism.

10. The method of claim 9, wherein the organism is a plant or an insect.

11. The method of claim 9, further comprising:
actuating, by the computing device, a mechanical device for moving a camera arm of the camera into an image capture orientation based at least in part on the triggering condition being detected, the camera arm being attached to the fixed structure.

12. The method of claim 9, wherein the organism is a weed plant and characteristic comprises at least one of: a weed species type, a quantity of weeds in the image; a quantity of weed leaves on a respective weed plant, or a growth stage of the respective weed plate.

13. The method of claim 9, wherein the triggering condition is a scheduled image capture time based at least in part on an interval time.

14. The method of claim 9, further comprising:
actuating, by the computing device, a mechanical device for moving a camera arm of the camera to an unobstructed orientation based at least in part on an occurrence of the capture of the image of the ecological environment having been completed, the camera arm being attached to the fixed structure.

15. A system, comprising:
a camera for capturing an image of an ecological environment;
a computing device that comprises a processor, and a memory, the computing device being in data communication with the camera; and
machine-readable instructions stored in the memory that, when executed by the processor, cause the computing device to at least:
identify a triggering condition for the ecological environment;
position the camera in a media capture orientation based at least in part the triggering condition;
capture, using the camera, an image of the ecological environment; and
determine a characteristic of an organism in the ecological environment based at least part in a machine learning model using an object detection technique.

16. The system of claim 15, wherein the machine-readable instructions that position the camera in a media capture orientation further cause the computing device to:
actuate a mechanical device for moving the camera to the media capture orientation.

17. The system of claim 15, wherein the triggering condition is a first triggering condition, and the machine-readable instructions, when executed, cause the computing device to at least:
actuate a mechanical device for moving the camera to a first orientation to a second orientation based at least in part on at least one of the capture of the image of the ecological environment or a second triggering condition.

18. The system of claim 17, wherein the mechanical device is a stepper motor or an actuator.

19. The system of claim 15, wherein the machine-readable instructions, when executed, cause the computing device to at least:
transmit the characteristic of the organism to a remote computer device based at least in part on a transmission condition.

20. The system of claim 15, wherein the computing device comprise a real-time clock device, wherein the triggering condition is based at least in part on an timing input from the real-time clock device.
